(19) United States
(12) Patent Application Publication
(10) Pub. No.: US 2006/0142251 A1
(43) Pub. Date: Jun. 29, 2006

(54) Anti-microbial agents derived from methionine sulfoximine analogues

(76) Inventors: Gunther Harth, Los Angeles, CA (US); Owen W Griffith, Milwaukee, WI (US); Marcus A Horwitz, Los Angeles, CA (US)

Correspondence Address: PRESTON GATES & ELLIS LLP, 1900 MAIN STREET, SUITE 600, IRVINE, CA, 92614-7319, US

(21) Appl. No.: 10/534,660

(22) PCT Filed: Nov. 17, 2003
(86) PCT No.: PCT/US03/36705

§ 371 Date: Nov. 28, 2005

Related U.S. Application Data
(60) Provisional application No. 60/426,502, filed on Nov. 15, 2002.
(60) Provisional application No. 60/430,407, filed on Dec. 2, 2002.

(51) Int. Cl. A61K 31/198; A61K 31/66

(52) U.S. Cl. 514/114; 514/562

(57) ABSTRACT

Novel antimicrobial compositions containing analogues of L-methionine-SR-sulfoximine (MSO) that are effective in treating intracellular pathogen infections are provided. Specifically, the compositions provided are MSO analogues having superior antimicrobial activity with significantly less toxicity as compared to MSO. These MSO analogues are suitable for use in treating infection in animals including primates, cows, pigs, horses, rabbits, mice, rats, cats, and dogs. Moreover, the MSO analogues are ideally suited for treating infections caused by the genus Mycobacterium. Additionally, methods for using the novel MSO analogues are also provided.

### RELATED APPLICATIONS

The present application claims priority to U.S. provisional patent application Ser. Nos. 60/426,502 filed Nov. 15, 2002, now abandoned and 60/430,407 filed Dec. 2, 2002, now abandoned both of which are incorporated herein in their entirety by reference.

### REFERENCE TO GOVERNMENT

This invention was made with Government support under Grant No. AI42925 awarded by the Department of Health and Human Services. The Government has certain rights in this invention.

### FIELD OF THE INVENTION

The present invention relates to anti-microbial agents useful in treating intracellular pathogen infections in animals. Specifically, the present invention relates to methionine sulfoximine (MSO) analogues and structurally similar compounds useful in treating intracellular pathogen infections. More specifically, the present invention relates to MSO analogues and structurally similar compounds useful in treating infections in animals caused by the genus Mycobacterium.

### REFERENCE CITATIONS

Reference to numerous articles and publications are made through the text. For convenience, each reference is cited using numerical notations enclosed in parentheses. These numerical notations correspond to the complete citation found in the Literature Cited list immediately preceding the Claims.

### BACKGROUND OF THE INVENTION

Mycobacterium tuberculosis is one of the world's most important and successful pathogens. It infects 2 billion persons worldwide, and it causes 8 million new cases of tuberculosis and 2 million deaths annually (1). Additionally, it is the leading cause of death in AIDS patients, whose susceptibility to tuberculosis is increased 100-fold. Compounding these problems, strains of M. tuberculosis resistant to conventional antibiotics used to treat the pathogen are rapidly emerging worldwide (2, 3). The rising worldwide incidence of tuberculosis and the rapid worldwide emergence of multidrug resistant strains of M. tuberculosis prompted the World Health Organization to declare tuberculosis a Global Emergency, the first disease so designated. Furthermore, multidrug resistant tuberculosis (MDRTB) strains are potential weapons of bioterrorism and have been classified as NIAID/CDC Category C Bioterrorism Agents. This has given new urgency to the need for novel strategies for combating M. tuberculosis.

Previously, the enzyme glutamine synthetase (GS) (E.C. 6.3.1.2) was identified as a potential antibiotic target (4, 5). In addition to its well-characterized role in nitrogen metabolism, in pathogenic mycobacteria, GS appears to play an important role in cell wall biosynthesis, providing substrate for the synthesis of a major poly L-glutamate/glutamine cell wall component found exclusively in pathogenic mycobacteria. Treatment of M. tuberculosis with either the GS inhibitor L-methionine-SR-sulfoximine (MSO) or with antisense oligodeoxyribonucleotides specific to M. tuberculosis GS mRNA inhibits formation of the poly-L-glutamate/glutamine cell wall structure (5, 6). Paralleling this effect, these agents also inhibit bacterial growth, indicating that the enzyme plays an important role in bacterial homeostasis (5, 6). MSO selectively blocks the growth of pathogenic mycobacteria in broth culture, including M. tuberculosis, M. bovis, and M. avium, but has no effect on nonpathogenic mycobacteria or nonmycobacterial microorganisms (5). The inhibitor also blocks the growth of M. tuberculosis and M. avium growing within human mononuclear phagocytes, the primary host cells of these pathogens, and at concentrations that are nontoxic to these mammalian cells, likely reflecting the 100-fold greater sensitivity to MSO of bacterial GS compared with mammalian GS (5).

The present inventors have examined the efficacy of MSO against M. tuberculosis in vivo. In guinea pigs challenged by aerosol with the highly virulent Erdman strain of M. tuberculosis, MSO administered once daily protected the animals against weight loss, a hallmark of tuberculosis, and against growth of M. tuberculosis in the lungs and spleen, reducing colony-forming units (CFU) of M. tuberculosis at 10 weeks after challenge by ˜0.7 logs compared with control animals. Importantly, MSO acted synergistically with isoniazid in protecting animals against weight loss and bacterial growth, reducing CFU in the lungs and spleen ˜1.5 logs below the level achieved with isoniazid alone.

The toxicity of MSO to mammals is generally attributed mainly to inhibition of GS and glutamine synthesis. However, in addition to inhibiting GS, MSO also inhibits γ-glutamylcysteine synthetase (γ-GCS), the rate limiting enzyme in glutathione synthesis. The resulting glutathione deficiency and consequent mitochondrial damage that results may contribute to the toxicity of MSO (7). Administration of ascorbate (Vitamin C) may offset this toxic effect of MSO, providing an alternative anti-oxidant and preserving glutathione levels (7, 8). This is a particularly important issue in guinea pigs because they, like humans, can not synthesize ascorbate. Consequently, the present inventors have investigated the impact of ascorbate on the maximum tolerated dose (MTD) of MSO in guinea pigs. In the presence of ascorbate, the MTD of MSO was 12.5 mg/kg/day, 4-fold higher than in the absence of ascorbate.

Furthermore, the impact on tuberculosis in guinea pigs of higher doses of MSO allowed by concomitant administration of ascorbate has also been studied. In the presence of ascorbate, the higher doses of MSO were highly efficacious. At the non-toxic dose of 6.25 mg/kg/day, treatment with MSO reduced CFU in the lungs and spleen by 2.5 logs compared with control animals. This level of reduction in the guinea pig as a result of treatment with MSO rivals the impact of isoniazid, the most potent antituberculosis drug. M. tuberculosis is the world's leading cause of death from a single infectious agent and the leading cause of death in AIDS patients.

During the past several years the present inventors have laid the groundwork for the development of a new antimicrobial strategy against M. tuberculosis—targeting M. tuberculosis glutamine synthetase (GS). See for example U.S. Pat. No. 6,013,660 issued to Horwitz, et al. Jan. 11, 2000 “Externally Targeted Prophylactic and Chemotherapeutic Method and Agents.” Thus, it has been demonstrated that M. tuberculosis GS is a promising antimicrobial target, and that the high production of this enzyme is correlated with pathogenicity in mycobacteria and with the presence of a poly-L-glutamate/glutamine structure in the cell wall of pathogenic mycobacteria. Horwitz et al. showed further that inhibition of GS with L-methionine-SR-sulfoximine (MSO) inhibits M. tuberculosis growth in cell-free culture, in human macrophages, and in vivo in guinea pigs challenged by aerosol with M. tuberculosis. In combination with ascorbate, MSO is almost as potent as isoniazid, the leading anti-tuberculous drug.

However, MSO is not an ideal therapeutic agent. First, as already noted, it inhibits γ-GCS. While this side effect can be minimized with respect to some glutathione functions by co-administration of Vitamin C, an analog of MSO lacking the capacity to inhibit γ-GCS would be preferable. Second, MSO is metabolized in vivo to form the corresponding keto acid and related products that break down spontaneously to form potentially toxic species including methane sulfinimide and vinylglyoxylate, a reactive Michael acceptor (13). Third and most importantly, MSO is a known epileptogenic agent (9). The sensitivity of various animal species to this effect of MSO varies greatly; dogs are highly sensitive (10) whereas humans are reportedly relatively insensitive to MSO. Although humans fed amounts of MSO-containing food that would cause toxicity in dogs exhibited no significant clinical, electroencephalographic, or biochemical abnormalities (9, 11), it would be preferable to have agents with diminished mammalian toxicity and thus a better therapeutic index. The epileptogenic effect of MSO is due to its inhibition of brain GS (8). Thus, analogs of MSO that are poorly transported into the brain and/or are even more specific for M. tuberculosis GS relative to brain GS than MSO would be highly desirable.

Therefore, there is a need for effective anti-mycobacterial therapeutic agents comprising GS inhibitors that meet the following three criteria: 1) the GS inhibitor must inhibit mycobaterial GS preferentially to mammalian GS; or not cross the blood-brain barrier at levels that inhibit mammalian GS to a clinically significant extent; 2) the GS inhibitor should not inhibit glutathione synthesis (i.e. not inhibit γ-GCS); 3) the GS inhibitor should not be metabolized into compounds toxic to mammals.

### SUMMARY OF THE INVENTION

The present inventors have discovered novel anti-mycobacterial compositions with reduced, or no toxicity to mammalian hosts. The invention is based on discovery that gamma-substituted derivatives of alpha-amino-alpha-alkyl-butyrates (see FIG. 6) effectively inhibit mycobacterial glutamine synthetase (MbGS), but do not substantially interfere with, or inhibit mammalian glutamine synthetase (MGS) in vivo partially due to the reduced ability of the alpha-substituted compounds to cross the blood-brain barrier. Moreover, the present inventors have discovered that where the alpha-alkyl substituent is two carbons or greater, MbGS is effectively inhibited but mammalian gamma-glutamylcysteine synthetase (γ-GCS) is not significantly inhibited and thus glutathione synthesis remains unaffected.

One embodiment of the present invention is an anti-mycobacterial composition comprising a gamma-substituted derivative of alpha-amino-alpha-alkyl-butyrate wherein the alpha alkyl group includes branched and straight-chained alkyl groups having from 2 to 8 carbons and the gamma substituent is a tetrahedral sulfur or phosphorus group.

In still another embodiment of the present invention the alpha alkyl group includes branched and straight-chained alkyl groups having from 2 to 4 carbons.

In another embodiment the tetrahedral sulfur or phosphorus group is selected from the group consisting of methyl sulfoximine, methyl sulfone, methyl sulfoxide, sulfonate, sulfonamide, phosphonate, methylphosphinite, phosphonamide.

In yet another embodiment of the present invention the anti-mycobacterial compostions of the present invention comprise alpha alkyl substituted L-methionine-SR-sulfoximine (MSO) wherein the alpha alkyl substituted MSO inhibits MbGS preferentially to MGS under in vivo conditions.

Therefore, it is an objective of the present invention to provide novel MSO analogs and structurally similar compounds that are useful in treating, palliating or inhibiting Mycobacterial disease progression in mammals.

It is another object of the present invention to provide novel MSO analogs and structurally similar compounds selective for glutamine synthetase that demonstrate reduced toxicity in animals compared to MSO.

It is yet another object of the present invention to provide novel MSO analogs and structurally similar compounds selective for glutamine synthetase that are useful in treating Mycobacterium sp. infections and demonstrate reduced toxicity in animals compared to MSO.

Toward this end the present inventors have developed novel MSO analogues (see FIG. 6) including, but not limited to, α-methyl-DL-methionine-SR-sulfoximine (α-Me-MSO) and α-ethyl-DL-methionine-SR-sulfoximine (α-Et-MSO). These exemplary MSO analogues are resistant to metabolism, and as a result do not form the toxic products that are formed in vivo from MSO (12, 13). While MSO, α-Me-MSO, and α-Et-MSO all inhibit mammalian GS, only MSO and α-Me-MSO inhibit γ-GCS (8). Thus, in contrast to MSO and α-Me-MSO, α-Et-MSO is specific to GS. Moreover, α-Et-MSO does not enter the brain as readily as MSO and is therefore much less likely to cause convulsions at therapeutic levels.

The MSO analogs and structurally similar compounds of the present invention are administered to animals including humans with active mycobacterial infection, e.g. infection with M. tuberculosis, M. bovis, or M. avium or people harboring M. tuberculosis in a latent state as evidenced by a positive diagnostic test for this organism. The MSO analogs and structurally similar compounds of the present invention may be formulated as pharmaceutical preparations using techniques known to those skilled in the art of medicinal chemistry and pharmaceutical formulations. The properly formulated compositions are then suitable for administration by any number of routes such as, but not limited to, intravenously, intramuscularly, intraperitoneally, subcutaneously, orally, and others. The MSO analogs and structurally similar compounds would inhibit the growth of pathogenic mycobacteria such as M. tuberculosis and thereby treat active tuberculosis or other mycobacterial infection or prevent latent tuberculosis from reactivating.

Other embodiments of the present invention include administering an anti-mycobacterial effective amount of MSO together with ascorbate (vitamin C) and co-administering the MSO analogues of the present invention with isoniazid (INH).

### BRIEF DESCRIPTION OF THE DRAWINGS

FIG. 1 graphically depicts weight loss and death after M. tuberculosis challenge and treatment with MSO and/or INH. The survival data in Experiment 1 are the percentage of animals surviving to the end of the 10-week observation period. In Experiment 2, all animals survived to the end of the observation period.

FIG. 2 graphically depicts growth of M. tuberculosis in the lung and spleen of guinea pigs after M. tuberculosis challenge. In Experiment 2, two lung cultures and four spleen cultures from the group treated with INH (4.0 mg kg⁻¹day⁻¹)+MSO (1.5 mg kg⁻¹ day⁻¹) had 0 CFU on plates seeded with undiluted samples. For statistical purposes, these organs were scored as 2.0 logs.

FIG. 3 graphically depicts MSO efficacy in the presence of ascorbate. Data are the mean net weight gain or loss ±SE for each group of animals compared with their weight just before challenge. (b). CFU in the lungs and spleens. Data are the mean±SE for all animals in a group.

FIG. 4 graphically depicts the in vitro antimicrobial activity of MSO, α-Me-MSO, and α-Et-MSO at final concentrations of 10, 100, and 1000 μM. Controls included no inhibitor or buthionine sulfoximine (BSO), a selective inhibitor of γ-GCS.

FIG. 5 graphically depicts the antimicrobial effects of MSO at 10, 100, and 1000 μM), and α-Me-MSO, and α-Et-MSO at 20, 200, or 2000 μM) against intracellular Mycobacterial infections in human macrophages (THP-1 cells).

FIG. 6 depicts GS inhibitors structurally related to MSO and used in accordance with the teachings of the present invention.

### DETAILED DESCRIPTION OF THE INVENTION

The present inventors have discovered novel anti-mycobacterial compositions with reduced, or no toxicity to mammalian hosts. The invention is based on discovery that gamma-substituted derivatives of alpha-amino-alpha-alkyl-butyrates effectively inhibit mycobacterial glutamine synthetase (MbGS), but do not substantially interfere with, or substantially inhibit, mammalian glutamine synthetase (MGS) in vivo partially due to the inability of the alpha-substituted compounds to cross the blood-brain barrier). As used herein the term “substantially interfere with” or “substantially inhibit” shall mean an MSO analogue of the present invention that, when administered to a mammalian host at a therapeutic dose, does not induce clinically significant toxic side effects including, but not limited to convulsions, associated with inhibition of mammalian GS. Persons having ordinary skill in the art of medicinal chemistry and physiology are able to easily ascertain when mammalian GS has been substantially inhibited or interfered with by observing the recipient's behavior or clinical signs and symptoms. Moreover, “substantially interfered with” and substantially inhibited” may be used interchangeably in the specification and claims and no significance is to be given to the different terms. Finally, “effectively inhibits MbGS” shall be defined to mean an anti-mycobacterial effective amount of the MSO analogue such that growth of the infecting mycobacterium is sufficiently suppressed, reduced or eliminated such that the disease process associated the infecting mycobacterium is clinically diminished. Clinically diminished shall mean a reduction in the disease state as measured or observed by a clinician having ordinary skill in the art of anti-microbial therapy or infectious diseases.

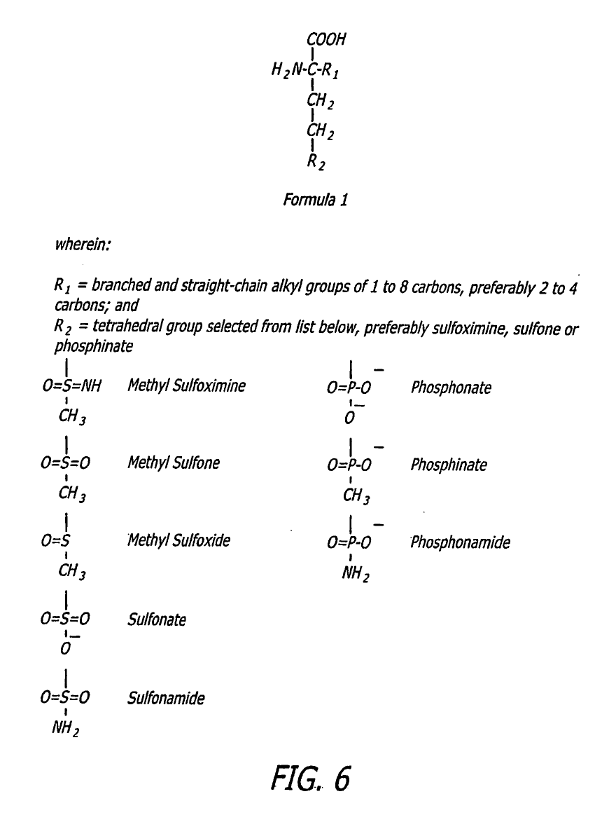

The present inventors have discovered that where the alpha-alkyl substituent is two carbons or greater, MbGS is effectively inhibited but mammalian gamma-glutamylcysteine synthetase (γ-GCS) is not inhibited and thus glutathione synthesis remains unaffected. Specifically, the present invention provides novel analogues of L-methionine-SR-sulfoximine (MSO) and structurally similar compounds that are effective in treating intracellular pathogen infections. More specifically, the present inventors have developed MSO analogues and structurally similar compounds having superior antimicrobial activity with significantly less toxicity as compared to MSO. The compositions of the present invention are suitable for use in treating infection in animals including primates, cows, sheep, horses, rabbits, mice, rats, cats and dogs. Moreover, the compositions of the present invention are ideally suited for treating infections caused by the genus Mycobacterium. Additionally, methods for using novel MSO analogues and structurally similar compounds are also provided. The novel MSO analogues of the present invention include, but are not limited to the compounds depicted in FIG. 6.

In view of the considerations discussed above, glutamine synthetase (GS) inhibitors that effectively inhibit mycobacterial GS without significant inhibition of mammalian γ-GCS would be desirable. Preferably, the GS inhibitors should inhibit mycobacterial GS at least as strongly as MSO. Furthermore, GS inhibitors that are less likely to be transported into the brain when used at therapeutic levels would be desirable.

The GS inhibitors of the present invention were tested for their ability to inhibit mammalian γ-GCS and for toxicity in mice. The GS inhibitors of the present invention were then tested for their capacity to inhibit the multiplication of M. tuberculosis in broth culture, in human macrophages and in guinea pigs.

Before testing was done in animals the present inventors determined the maximum tolerated dose (MTD) of MSO in uninfected and then in infected guinea pigs. Next efficacy of the compounds in treating M. tuberculosis infection in guinea pigs was determined by assessing the impact of the drug on weight change after aerosol infection with M. tuberculosis, survival after infection, and CFU in the lungs and spleen. As a potential therapeutic agent, MSO has three major drawbacks. First, as already noted, it inhibits γ-GCS. While it has been discovered that this side effect can be minimized with respect to some glutathione functions by co-administration of Vitamin C, an analog of MSO lacking the capacity to inhibit γ-GCS would be preferable. Second, MSO is metabolized in vivo to form the corresponding keto acid and related products that break down spontaneously to form potentially toxic species including methane sulfinimide and vinylglyoxylate, a reactive Michael acceptor (13). Third and most importantly, MSO is a known epileptogenic agent (9). The sensitivity of various animal species to this effect of MSO varies greatly; dogs are highly sensitive (10) whereas humans are reportedly relatively insensitive to MSO. Although humans fed MSO-containing food that would cause toxicity in dogs exhibited no significant clinical, electroencephalographic, or biochemical abnormalities (9, 11), it would be preferable to have agents with diminished mammalian toxicity. The epileptogenic effect of MSO is due to its inhibition of brain GS (8). Thus, analogs of MSO that are poorly transported into the brain and/or are even more specific for M. tuberculosis GS relative to brain GS than MSO would be highly desirable.

The present inventors selected two exemplary compounds for testing: α-methyl-DL-methionine-SR-sulfoximine (α-Me-MSO) and α-ethyl-DL-methionine-SR-sulfoximine (α-Et-MSO). Both compounds are resistant to metabolism, and as a result they do not form the toxic products that are formed in vivo from MSO (12, 13). While MSO, α-Me-MSO, and α-Et-MSO all inhibit mammalian GS, only MSO and α-Me-MSO inhibit γ-GCS (8). Thus, in contrast to MSO and α-Me-MSO, α-Et-MSO is specific to GS. Moreover, α-Me-MSO and α-Et-MSO do not enter the brain as readily as MSO. While all three compounds cause convulsions, the dose of α-Et-MSO that induces convulsions in a minority of mice is 16-fold higher than the dose of MSO that induces convulsions in 100% of mice (8). Thus α-Et-MSO is less toxic for mammals than MSO. Similarly, the dose of α-Me-MSO that causes convulsions in mice is ˜8 fold higher than MSO. Exemplary, non-limiting GS inhibitors of the present invention and their relevant properties are summarized below:

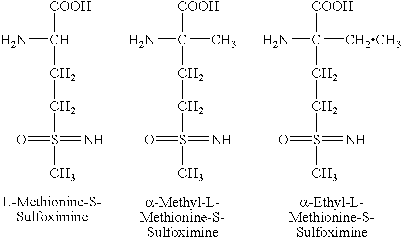

TABLE 1

| | | | | | Estimated Dose |
|---|---|---|---|---|---|
| | | | | | of L Isomer |
| | | | | | that Induces |
| | | | | | Convulsions |
| | | | | | in Mice |
| | Inhibits | Inhibits | Inhibits | Metabolized | (Relative |
| Inhibitor | MbGS | MGS | γ-GCS | In Vivo | to MSO) |
| MSO | + | + | + | + | 1 |
| α-Me- | + | + | + | − | 8 |
| MSO | | | | | |
| α-Et-MSO | + | + | − | − | >16 |

The present inventors tested the efficacy of these analogs of MSO against M. tuberculosis in broth culture and their inhibitory capacity was comparable to or greater than that of MSO. The compounds were also inhibitory to M. tuberculosis in macrophages. Thus, while the α-ethyl group reduces the transport of MSO into the brain, it apparently does not influence its transport into M. tuberculosis.

Bacterial and mammalian GS catalyze an identical chemical reaction (Reaction 1) and both of their active sites contain two catalytically essential Mg²⁺ ions (Mn²⁺ is also active) (27, 36). The bacterial and mammalian GS differ in quarternary structure (dodecamer vs. octomer, respectively), and comparison of the deduced amino acid sequences of mycobacterial and human GS shows only a low degree of overall identity (˜24%) and similarity (31%). Very recently, J. J. Abbott et al. (40) were able to use the known X-ray crystallographic structure of Salmonella typhimurium GS (36) and sophisticated sequence comparison software to tentatively identify conserved amino acids that serve as Mg²⁺-binding ligands in the active sites of all GS for which full sequence information is available. In that analysis, mycobacterial GS and human GS were assigned to the distinct GS families I and II, respectively, and there was only limited additional sequence similarity even among the residues closely adjacent to the conserved Mg²⁺-binding ligands. Absence of significant sequence similarity between mycobacterial and human GS makes it unpredictable whether known inhibitors of mammalian (human) GS will also inhibit mycobacterial GS (or vice verse). Ideally, inhibitors selective for mycobacterial GS over mammalian GS would be discovered. For two inhibitors, MSO and phosphinothricin, it is established that a selectivity for mycobacterial over mammalian GS of at least 100-fold exists.

Recently obtained high resolution X-ray crystallographic structure of the M. tuberculosis GS (41) indicates that the 3-dimensional structure of mycobacterial GS is very similar to the previously determined S. typhimurium GS structure (36). Although the M. tuberculosis GS structure is of the “relaxed” conformation that does not bind substrates or inhibitors, the now documented close similarity between the Salmonella and Mycobacterium GS structures aids inhibitor design efforts. In particular, X-ray crystallographic structures of MSO and related inhibitors bound to Salmonella GS (19, 20, 36) are depicted that provide some of the basis for the discovery by the present inventors of novel structurally similar inhibitors and provide a basis for additional modifications.
Reaction 1: L-Glutamate+NH₃+ATP→L-Glutamine+ADP+Pi

In addition to MSO, mycobacterial and/or mammalian GS are inhibited by a number of structurally related MSO analogs including methionine sulfoxide and methionine sulfone and by structurally related phosphorous-containing derivatives such as phosphinothricin. Each of these compounds places a tetrahedral sulfur or phosphorous moiety in the part of the GS active site normally occupied by the γ-carboxylate of substrate L-glutamate. Structurally related inhibitors including the sulfonate, sulfinate and sulfonamide that are structurally related to MSO and the phosphonate, phosphinate and phosphonamide that are related to phosphinothricin (see FIG. 6) similarly bind to the active site of and thereby inhibit GS. Such compounds are also known or likely inhibitors of γ-GCS. As with MSO, selectivity for GS over γ-GCS, diminished uptake into brain, and/or diminished mammalian metabolism to toxic species are achieved with these related inhibitors by the structural modification of adding an α-alkyl substituent of 1 to 8 carbons, preferably an α-alkyl substituent of 2 to 4 carbons (ethyl, propyl, isopropyl, butyl, isobutyl, sec-butyl.

### EXAMPLES

### Example 1

### Determination of the Maximum Tolerated Dose (MTD) of MSO in Guinea Pigs

MSO was delivered i.p. to guinea pigs for 21 days and the animals were observed for weight loss and other adverse effects. Doses >12.5 mg kg⁻¹day⁻¹ were 100% lethal; 6.25 mg kg⁻¹ day⁻¹ was 33% lethal and otherwise poorly tolerated, inducing lethargy and anorexia; and doses #3 mg kg⁻¹ day⁻¹ were nonlethal (Table 1). In a subsequent experiment, Horwitz et al. determined that the dose of 3.0 mg kg⁻¹ day⁻¹ was well-tolerated by uninfected guinea pigs, but not by guinea pigs infected with M. tuberculosis, which exhibited early weight loss. Possibly, MSO's known negative impact on glutathione synthesis in the absence of ascorbate (see below) reduced the capacity of the animals to counter the stress of infection. In the infected animals, 1.5 mg kg⁻¹ day⁻ was well-tolerated and hence this dose was judged to be the maximum tolerated dose for guinea pigs infected with M. tuberculosis.

TABLE 2
Maximum Tolerated Dose of MSO in Guinea Pigs ± Ascorbate

| | MSO (mg kg⁻¹ day⁻¹) Alone | | | | | | | MSO (mg kg⁻¹ day⁻¹) + Ascorbate | | | | |
|---|---|---|---|---|---|---|---|---|---|---|---|---|
| | 100 | 50 | 24 | 12.5 | 6.25 | 3.13 | 1.56 | 25 | 12.5 | 6.25 | 3.13 | 1.56 |
| Number of Animals | 3 | 3 | 5 | 2 | 3 | 3 | 3 | 3 | 3 | 3 | 3 | 3 |
| Deaths (%) | 100 | 100 | 100 | 100 | 33 | 0 | 0 | 100 | 0 | 0 | 0 | 0 |
| Median Time to Death (days) | 1 | 2 | 2.5 | 4 | 5 | — | — | 6 | — | — | — | — |

### Example 2

### Demonstration that MSO Protects Guinea Pigs from Death and Disease

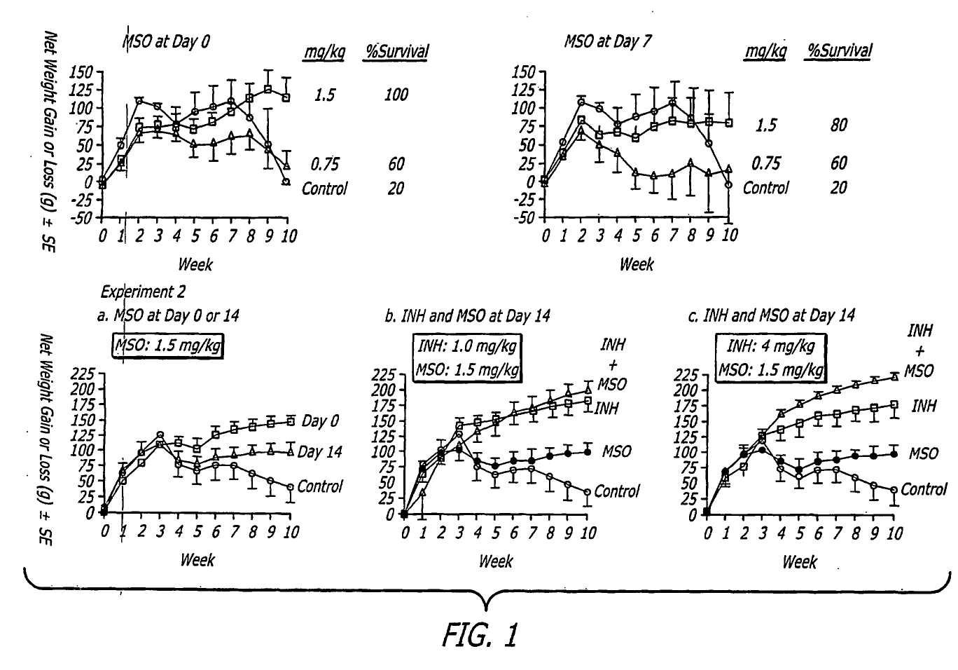

The present inventors infected guinea pigs in groups of 5 by aerosol with the highly virulent Erdman strain of M. tuberculosis, administered MSO to the animals at doses of 1.5 or 0.75 mg kg⁻¹ day⁻¹ i.p. for 10 weeks beginning immediately or 7 days after challenge, and monitored the subsequent course of infection. Control animals were untreated. Death is not an endpoint in the majority of such studies because untreated guinea pigs usually do not succumb to tuberculosis until after 10 weeks following challenge, the point at which the study is terminated. However, deaths do occasionally occur earlier than 10 weeks, and this was the case in the present study. Whereas almost all of the animals treated with 1.5 mg kg⁻¹ day⁻¹ MSO (n=10) survived the 10 week observation period, whether treatment was begun on day 0 (100% survival) or day 7 (80% survival) after challenge, only 20% of the control animals survived (n=5) (FIG. 1, Experiment 1). Animals treated with 0.75 mg kg⁻¹ day⁻¹ MSO (n=10) were partially protected against death; 60% of these animals survived whether treatment was initiated on day 0 or 7 after challenge. Differences in survival were statistically significant between untreated controls and a) animals treated with MSO 1.5 mg kg⁻¹ day⁻¹ beginning on day 0 (P<0.05, Chi Square Statistic); b) animals treated with MSO 1.5 mg kg⁻¹ day⁻¹ beginning on day 0 or day 7 (P<0.02); and c) animals treated with MSO 1.5 mg kg⁻¹ day⁻¹ or MSO 0.75 mg kg⁻¹ day⁻¹ beginning on day 0 or day 7 (P<0.05).

An objective indicator of illness is weight loss, a major physical sign of tuberculosis in humans and a hallmark of the disease in the guinea pig model of this chronic infectious disease. Compared with untreated control animals, animals treated with 1.5 mg kg⁻¹ day⁻ MSO were protected from weight loss in the final weeks of the observation period, by which time the disease in control animals was far-advanced. Differences in net weight gain between MSO-treated and control animals were statistically significant when treatment was begun immediately after challenge (P=0.02, Experiment 1). Animals treated with 0.75 mg kg⁻¹ day⁻¹ had lower weight gain than controls in the first 9 weeks after challenge, but were protected from a precipitous decline in weight loss in the final week of the observation period (FIG. 1, Experiment 1). Animals treated with 1.5 or 0.75 mg kg⁻¹ day⁻¹ MSO immediately after challenge gained slightly more weight than animals treated with the same doses beginning 7 days after challenge, but the survival rates were comparable. Similarly, in a second experiment, animals treated immediately after challenge gained more weight than animals in which treatment was begun 14 days after challenge (FIG. 1, Experiment 2, leftmost panel). Differences in net weight gain between MSO-treated and control animals were statistically significant whether MSO was started immediately (P=0.003) or 14 days after challenge (P=0.04) (Experiment 2a).

FIG. 1 graphically depicts weight loss and death after M. tuberculosis challenge. Animals in groups of 5 were infected with M. tuberculosis and treated with MSO and/or INH beginning 0, 7, or 14 days after challenge, as indicated, or not treated (controls). All animals were weighed weekly for 10 weeks after challenge and monitored for survival. Weight data are the mean net weight gain or loss ±SE for each group of animals compared with their weight immediately before challenge. The survival data in Experiment 1 are the % of animals surviving to the end of the 10-week observation period. In Experiment 2, all animals survived to the end of the observation period.

### Example 3

### Demonstration that MSO Inhibits Growth of M. Tuberculosis In Guinea Pig Lungs and Spleen

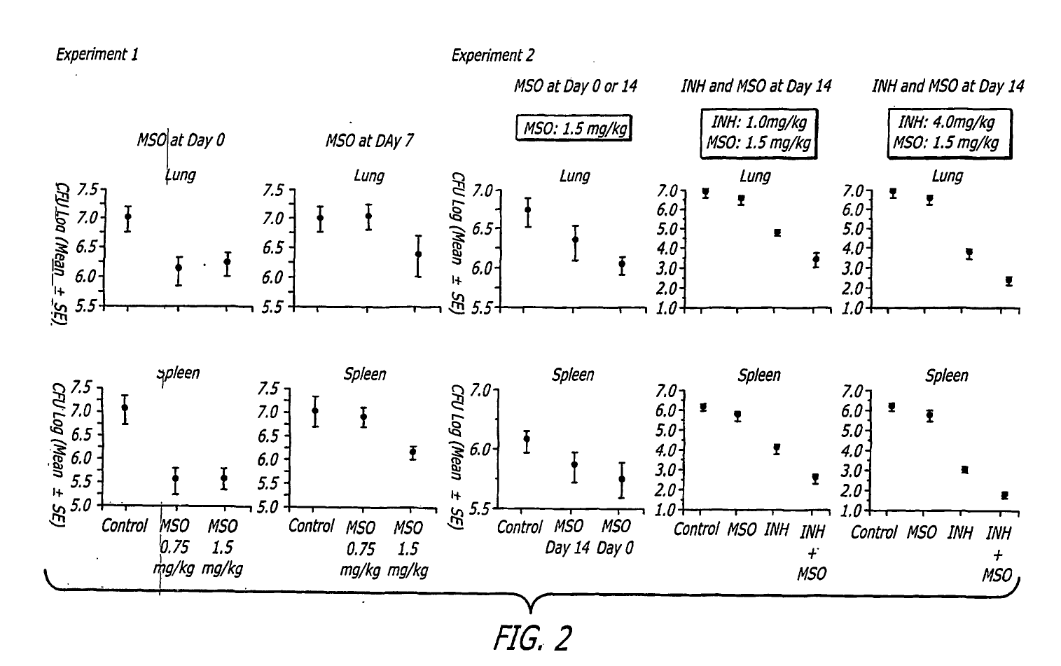

To assess the capacity of MSO treatment to restrict the growth of M. tuberculosis in tissues of challenged guinea pigs, Horwitz et al. assayed the number of bacteria in the lungs, the primary site of infection, and spleen, a major site of bacterial dissemination, at the end of the 10 week observation period. Animals treated with either 1.5 mg kg⁻¹ days⁻¹ MSO beginning on day 0 or day 7 after challenge, or with 0.75 mg kg⁻¹ days⁻¹ beginning on day 0 after challenge had approximately 1 log unit fewer CFU of M. tuberculosis in their organs than control animals (FIG. 2, Experiment 1), differences that were statistically significant and highly so in the spleen. When treatment was begun on day 0, doses of MSO of 0.75 and 1.5-mg kg⁻¹ days⁻¹ yielded comparable reductions in CFU. However, when treatment was delayed until day 7 after challenge, only the higher dose of MSO was effective in reducing bacterial counts in the lung and spleen. In a second experiment, MSO at 1.5 mg kg⁻¹ day⁻¹ was effective at reducing bacterial counts whether initiated at day 0 or 14 after challenge, although early initiation was somewhat more efficacious (FIG. 2, Experiment 2, leftmost panel) and the differences from controls were statistically significant.

FIG. 2 graphically depicts growth of M. tuberculosis in the lung and spleen of guinea pigs after M. tuberculosis challenge. At the end of the observation period, the animals described in FIG. 2 were euthanized and CFU of M. tuberculosis in the right lung and spleen were assayed. The few animals that died before the end of the observation period were cultured immediately after death. Data are the mean±SE for all animals in a group. The lower limit of detection was 2.0 log units per organ (I CFU on a plate seeded with an undiluted 1% sample of an organ, i.e. 100 μl of a total sample volume of 10 ml). In Experiment 2, two lung cultures and four spleen cultures from the group treated with INH (4.0 mg kg⁻¹ days⁻¹)+MSO (1.5 mg kg⁻¹ day⁻¹) had 0 CFU on plates seeded with undiluted samples. For statistical purposes, these organs were scored as 2.0 logs.

### Example 4

### Demonstration That MSO Acts Synergistically with Isoniazid to Protect Guinea Pigs Infected with M. tuberculosis

Tuberculosis in humans is generally treated with a combination of antibiotics to gain better control over the infection and to prevent the emergence of resistant organisms. To determine the efficacy of MSO in combination with another antituberculosis drug, Horwitz et al. studied the efficacy of MSO in combination with the major antituberculosis drug isoniazid (INH), which they had previously found acts synergistically with MSO against M. tuberculosis in broth culture (5). Horwitz et al. studied orally administered INH at the maximally effective dose in guinea pigs of 4 mg kg⁻¹ day⁻¹ and at the slightly less effective dose of 1 mg kg⁻¹ day⁻¹. The drugs were administered beginning 14 days after challenge, a point at which animals begin to exhibit signs of disease. Administered alone, both MSO and INH protected animals from weight loss; INH was more effective at both doses studied (FIG. 2, Experiment 2, center and rightmost panels). INH in combination with MSO yielded even greater protection against weight loss than INH alone, especially at the higher dose of INH, where the difference was statistically significant. Weight gain in animals treated with the two drugs in combination approximated that observed in uninfected animals of similar age in previous experiments (14). Paralleling these results, both MSO and INH reduced CFU of M. tuberculosis in the lung and spleen. INH was much more effective than MSO. At a dose of 1 mg kg⁻¹ day⁻¹, INH reduced CFU in the lung and spleen by ˜1.5 logs below the level obtained with MSO, and at a dose of 4 mg kg⁻¹day⁻¹, INH reduced CFU by ˜2.5 logs below the level obtained with MSO (FIG. 2, Experiment 2, center and rightmost panels). However, when the two drugs were administered in combination, they reduced CFU in the lung and spleen even further, ˜1.5 logs below the level obtained with INH alone, differences that were statistically highly significant in both organs at both doses of INH (P<0.0001, Experiment 2). Indeed, in animals treated with MSO plus the higher dose of INH (4.0 mg kg⁻¹ day⁻¹), the reduction in CFU was even greater than that indicated in FIG. 2; CFU were actually undetectable in the lungs of two animals and the spleens of four animals out of the five animals in this group, but scored as 2.0 logs, the lower limit of detection.

The findings at necropsy mirrored these results. On visual inspection, the left lungs and livers (these organs are preserved at necropsy) of animals treated with MSO, INH, and the combination of MSO and INH had progressively fewer lesions than untreated controls. Lesions were particularly scarce in the lungs and livers of animals treated with the combination of MSO and INH.

Thus, the combination of MSO and INH was more efficacious than either drug alone in protecting animals from disease, as reflected by weight loss, and the two drugs acted synergistically to suppress the growth of M. tuberculosis in the lung and spleen.

### Example 5

### Demonstration That MSO is Highly Efficacious as a Single Agent at Higher Doses Tolerated by Concomitant Administration of Ascorbate.

In addition to inhibiting GS, MSO also inhibits γ-GCS, the rate-limiting enzyme in glutathione synthesis. The glutathione deficiency that results might contribute to the toxicity of MSO. Griffith et al. have previously shown that glutathione depletion induced by administration of buthionine sulfoximine (BSO), a highly selective inhibitor of γ-GCS, is highly toxic to guinea pigs, killing 100% of animals at doses that fully inhibit glutathione synthesis (14 and J. Han and O. W. Griffith, unpublished observations). It was shown that ascorbate (Vitamin C) can offset this toxic effect of BSO and MSO and preserve critical mitochondrial glutathione levels (7, 8, 15).

To determine the maximum tolerated dose (MTD) of MSO in guinea pigs in the presence of ascorbate, Horwitz et al. administered MSO to animals at various doses in the presence of 3 mmoles kg⁻¹day⁻¹ ascorbate i.p. delivered in two equally divided doses. A preliminary study determined that a total dose of 3 mmoles kg⁻¹ day⁻¹ ascorbate i.p. but not 9 mmoles kg⁻¹ day⁻¹ was well tolerated by guinea pigs. In the presence of ascorbate, the MTD of MSO was 12.5 mg kg⁻¹ day⁻¹, 4-fold higher than in the absence of ascorbate (Table 2).

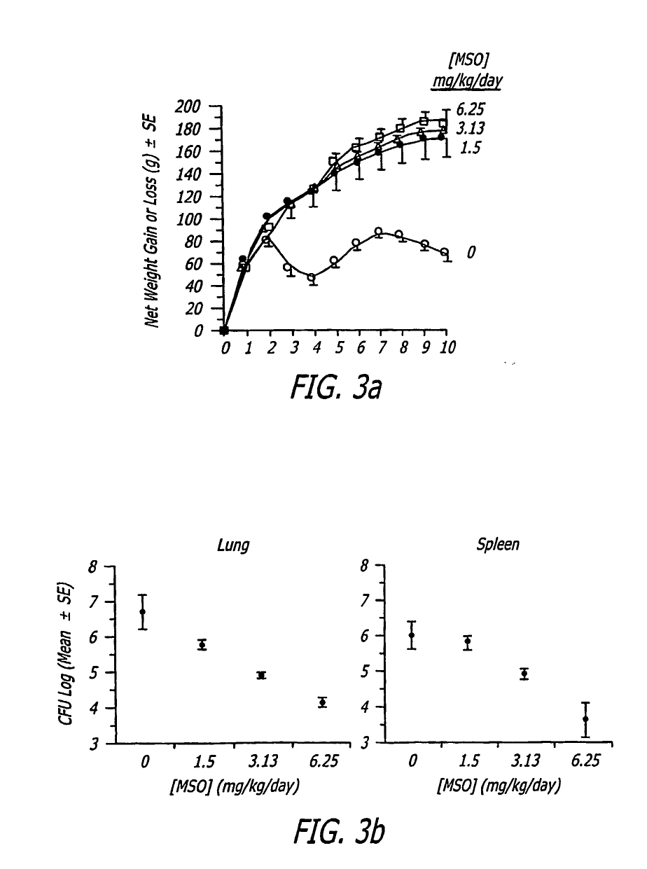

To determine the efficacy of the higher doses of MSO that are tolerated in the presence of ascorbate, Horwitz et al. infected guinea pigs with M. tuberculosis, and then treated them with MSO at concentrations of 0, 1.5, 3.13, and 6.25 mg kg⁻¹ day⁻¹ i.p in the presence of 3 mmoles kg⁻¹ day⁻¹ ascorbate i.p. starting immediately after challenge. As in previous experiments, the animals were observed for 10 weeks and then euthanized so that CFU in the lungs and spleen could be determined. All animals survived the observation period except for one untreated control animal (0 mg kg⁻¹ day⁻¹ MSO) that died 3.5 weeks after challenge. Animals administered MSO at each of the three doses gained significantly more weight than untreated controls (FIG. 3a). MSO-treated animals also had significantly fewer CFU in the lungs and spleen (FIG. 3b). The effect of MSO on CFU was strongly dose-dependent with 6.25>3.13>1.5 mg kg⁻¹days⁻¹. At the highest dose, 6.25 mg kg⁻¹days⁻¹, CFU were reduced 2.5 logs in the lungs and in the spleens compared with untreated controls, a highly significant difference (P<0.0001 for both organs).

FIG. 3 graphically depicts the efficacy of MSO in the presence of ascorbate. Animals in groups of 5 were infected with M. tuberculosis by aerosol and then treated with MSO at the concentrations indicated in the presence of ascorbate (3 mmoles/kg/day) for 10 weeks (a) Weight data. All animals were weighed weekly for 10 weeks. Data are the mean net weight gain or loss±SE for each group of animals compared with their weight just before challenge. (b). CFU in the lungs and spleens. Data are the mean±SE for all animals in a group.

### Example 6

### Demonstration that Alpha-Alkyl Analogs of MSO with Greatly Reduced Mammalian Toxicity Inhibit M. Tuberculosis Growth as Effectively as MSO

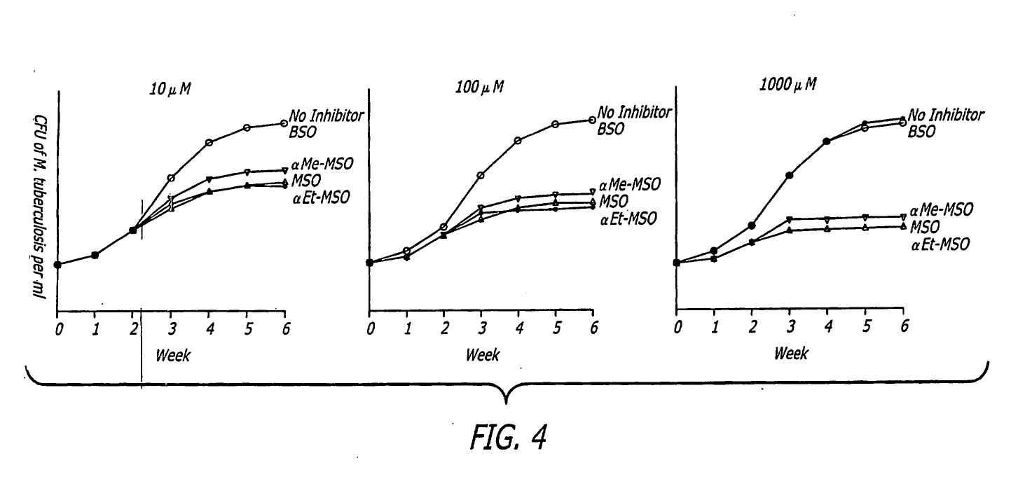

The present inventors tested the efficacy against M. tuberculosis growth in broth culture of three concentrations of α-methyl-DL-methionine-SR-sulfoximine (α-Me-MSO) and α-ethyl-DL-methionine-SR-sulfoximine α-Et-MSO) in comparison with MSO (FIG. 4). In all cases, only the L-S-diastereomer is an active GS inhibitor, and the concentration of active isomer in the α-alkyl-MSO derivative preparations is thus only 50% of that in the MSO (L-methionine-SR-sulfoximine) preparation used. Nevertheless, the α-Me-MSO and α-Et-MSO preparations were as inhibitory to M. tuberculosis growth as MSO.

FIG. 4 depicts a study in which MSO, α-Me-MSO, and α-Et-MSO at final concentrations of 10, 100, and 1000 μM were added to triplicate cultures of M. tuberculosis Erdman (maintained in Middlebrook 7H9 medium supplemented with 2% glucose at 37° C. in a 5% CO₂-95% air atmosphere) at a cell density of 1-5×10⁵ cells ml⁻¹ in tissue culture flasks. Growth of the cultures was monitored weekly for 6 weeks by gently sonicating to break up bacterial clumps, removing small aliquots, plating serial dilutions of washed bacteria on Middlebrook 7H11 agar, and enumerating CFU after incubation for 2 weeks. Controls included no inhibitor or buthionine sulfoximine (BSO), a selective inhibitor of γ-GCS.

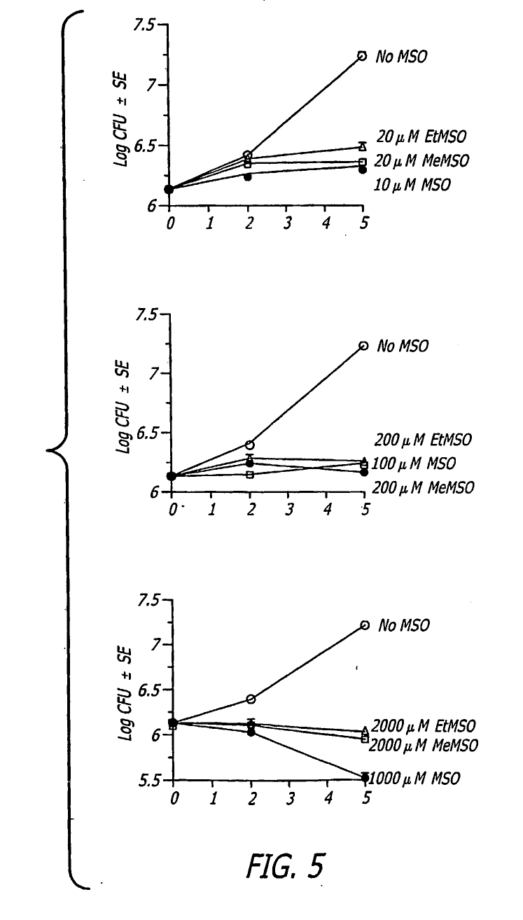

The α-Me-MSO and α-Et-MSO preparations also readily inhibited M. tuberculosis growth in human macrophages. The extent of inhibition was comparable to that of MSO at equivalent concentrations of the active isomer, except at the very highest dose tested where MSO inhibition was somewhat greater (FIG. 5).

FIG. 5. graphically depicts the results of a study with human macrophages (THP-1 cells) that were infected with M. tuberculosis Erdman and then treated with MSO at a final concentration of 10, 100, or 1000 μM or with an equivalent concentration, normalizing for the active isomer, of α-Me-MSO or α-Et-MSO (20, 200, or 2000 μM, respectively) as indicated. CFU were determined at 3 hours (Day 0), 2 days, and 5 days after infection. Data are the mean±SE for duplicate cultures.

### Example 7

### Capacity Of Various Concentrations Of MSO, α-Et-MSO, α-Me-MSO and D, L-Buthionine-SR-Sulfoximine (BSO) to Inhibit the Growth of M. Tuberculosis

This experiment tested the capacity of four compounds that inhibit glutamine synthetase and/or γ-glutamylcysteine synthetase to inhibit the growth of M. tuberculosis in broth culture. The inhibitors and their relevant properties are summarized in Table 3.

| | | | Estimated Dose of L |
|---|---|---|---|
| | Inhibits | Inhibits γ- | Isomer that Induces |
| | Glutamine | Glutamylcysteine | Convulsions in Mice |
| Inhibitor | Synthetase | Synthetase | (Relative to MSO) |
| MSO | + | + | 1 |
| α-Me-MSO | + | + | 8 |
| α-Et-MSO | + | − | ≧16 |
| BSO | − | + | N/A |

N/A: Not applicable; does not cause convulsions

M. tuberculosis Erdman strain was grown in 7H9 medium containing 2% glucose to an Optical Density (O.D.) (540 nm) of 0.5, sonicated, diluted in 7H9 medium to an O.D. of approximately 0.05, and 2 ml of the suspension added to triplicate 12×75 (5 ml) polystyrene test tubes. MSO, α-Et-MSO, α-Me-MSO, BSO at concentrations of 10, 100, or 1000 μM or buffer control (PBS) were added to the tubes. The cultures were incubated for 6 weeks. Colony-forming units of M. tuberculosis were assayed weekly by removing aliquots from the tubes, serially diluting, plating on 7H11 agar, and counting the colonies that formed after 2-weeks incubation at 37° C. in a 5% CO₂-95% air atmosphere.

MSO, α-Me-MSO, and α-Et-MSO, all of which inhibit glutamine synthetase, all inhibited M. tuberculosis, whereas BSO, which does not inhibit glutamine synthetase, did not. The magnitude of the inhibition by MSO, α-Et-MSO, and α-Me-MSO was dose-dependent (1000 μM>100 μM>10 μM). At each of the three concentrations, α-Et-MSO was as inhibitory or slightly more inhibitory than MSO, even though α-Et-MSO was a diastereomeric mixture of four isomers, whereas MSO was a diastereomeric mixture of two isomers. Only one of the isomers of each drug (the L-S-isomer) is likely to be active. At each of the three concentrations, α-Me-MSO was slightly less inhibitory than MSO, a difference that may have reflected the fact that α-Me-MSO was a mixture of four isomers whereas MSO was a diastereomeric mixture of two isomers. Again, only one of the isomers of each drug (the L-S-isomer) is likely to be active.

### Example 8

### Synthesis Of Alpha-Alkyl Analogs of MSO

α-Ethyl-DL-methionine, the precursor needed to make α-Et-MSO, was prepared by standard Bucherer amino acid synthesis from ethyl 2-(methylthio) ketone, ammonium bicarbonate and sodium cyanide; the required ketone was made by addition of methane thiol to ethyl vinyl ketone (42). The overall yield was 42%. α-Ethyl-DL-methionine was converted to the corresponding sulfoximine (i.e., α-Et-MSO) using sodium azide in chloroform and sulfuric acid (42). The sulfoximine was isolated in 65-75% yield. This general method is useful for preparing any α-substituted methionine and MSO analogue by replacing ethyl vinyl ketone with a vinyl ketone containing the desired α-substitutent in place of the ethyl moiety. In addition, a wide range of α-substituted L-methionine derivatives can be synthesized from commercially available D-methionine by the general method of Fadel and Salaun (43) for preparation of α-alkyl-L-methionine derivatives by direct alkylation of D-methionine phenyloxazolidinones. The 4 step procedure results in inversion of configuration at the α-carbon and provides α-alkyl-L-methionines in ˜95% yield and >95% enantiomeric purity. Because the Fadel and Salaun procedure yields nearly pure L-isomers, conversion to the corresponding sulfoximines yields diastereomeric mixtures of two isomers (e.g., α-alkyl-L-methionine-SR-sulfoximine.) Because only the L-S-diastereomer is biologically active, that active isomer can be isolated from either 2 isomer or 4 isomer mixtures by chiral HPLC techniques, enzymatic techniques, recrystallization techniques or by combinations of those techniques as is well known in the literature (45, 46).

Synthesis of the other derivatives is easily achieved by methods well known in the art. Specifically, the parent of each of the derivatives in which the alpha substituent is a proton rather than alkyl is well known in the chemical literature. Application of the general method of Fadel and Salaun (43) to those parent compounds or to precursors of the parent compounds provides a straight-forward synthetic route.

In all of the compounds of interest the alpha carbon is chiral. In all cases, it is the L-isomer that is biologically active and the invention is meant to embrace both the pure L-isomer(s) and mixtures that include the L-isomer(s) including racemic mixtures. Where the tetrahedral sulfur or phosphorous gamma-substituent is also chiral, the invention is meant to embrace the pure active isomer as well as mixtures that include the active isomer including racemic mixtures. Thus, for α-Et-MSO the active isomer is α-ethyl-L-methionine-S-sulfoximine, and that agent could be isolated (see above) and used in pure form, or as the diastereomeric mixture α-ethyl-L-methionine-R,S-sulfoximine, or as the 4 isomer mixture, α-ethyl-D,L-methionine-R,S-sulfoxinine.

Persons having ordinary skill in the art of infectious disease medicine and clinical microbiology can easily establish the antimicrobial effective amount of the compostions of the present invention. The examples provided herein teach methods for establishing an antimicrobial effective amount of the MSO analogs and structurally similar compounds of the present invention using in vitro and in vivo techniques. Based on these methods, and other methods known to those skilled in the art, an effective amount of an MSO analog or structurally similar compound appropriate for an animal's weight and body composition can be easily determined. Moreover, standard laboratory minimum inhibitory concentration (MIC) testing can be used to determine the relative susceptibility of individual intracellular pathogen strains isolated from an infected animal. The antimicrobial effective amount for any individual strain can then be determined using the MIC value thus obtained.

The present invention is useful for treating or preventing infections caused by Mycobacterium tuberculosis, the agent of tuberculosis, and infections caused by other pathogenic mycobacteria. New antibiotics are needed against this pathogen, which is rapidly developing resistance to conventional antibiotics worldwide. Therefore, the present invention provides a significant advantage over present antimicrobial therapies including MSO and provides a new type of antibiotic to treat infections caused by both drug resistant and drug sensitive strains of intracellular pathogens including M. tuberculosis and other pathogenic mycobacteria.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and an” and “the” and similar referents used in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventors expect skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety. Moreover, the specific sections of the specification or publication that is to be incorporated by reference is limited to that section, or sections, making specific reference to the topic under discussion where the reference is cited.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### LITERATURE CITED

1. C. Dye, S. Scheele, P. Dolin, V Pathania, M. C. Raviglione, 1999. Consensus statement. Global burden of tuberculosis: estimated incidence, prevalence, and mortality by country. WHO Global Surveillance and Monitoring Project. J. Am. Med. Ass. 282(7): 677-686.
2. Pablo-Mendez, A., M. C. Raviglione, A. Laszlo, N. Binkin, H. L. Rieder, F. Bustreo, D. L. Cohn, D. L., C. S. B Lambregts-van Weezenbeek, S. J. Kim, P. Chaulet, and P. Nunn. 1998. Global surveillance for antituberculosis-drug resistance, 1994-1997. New Engl. J. Med. 338:1641-1649.
3. Cohn, D. L., F. Bustreo, and M. C. Raviglione. 1997. Drug-resistant tuberculosis: review of the worldwide situation and the WHO/IUATLD Global Surveillance Project. International Union Against Tuberculosis and Lung Disease. Clin. Infect. Dis. 24:S121-130.
4. Harth, G., D. L. Clemens, and M. A. Horwitz. 1994. Glutamine synthetase of Mycobacterium tuberculosis: extracellular release and characterization of its enzymatic activity. Proc. Natl. Acad. Sci. USA. 91:9342-9346.
5. Harth, G. and M. A. Horwitz. 1999. An inhibitor of exported Mycobacterium tuberculosis glutamine synthetase selectively blocks the growth of pathogenic mycobacteria in axenic culture and in human monocytes: Extracellular proteins as potential novel drug targets. J. Exp. Med. 189:1425-1435.
6. Harth, G., P. C. Zamecnik, J-Y. Tang, D. Tabatadze, and M. A. Horwitz. 2000. Treatment of Mycobacterium tuberculosis with antisense oligonucleotides to glutamine synthetase mRNA inhibits glutamine synthetase activity, formation of the poly-L-glutamine/glutamate cell wall structure, and bacterial replication. Proc. Natl. Acad. Sci. USA. 97:41 8-423.
7. Meister, A. 1995. Mitochondrial changes associated with glutathione deficiency. Biochim. Biophys. Acta. 1271:35-42.
8. Griffith, O. W. and A. Meister. 1978. Differential inhibition of glutamine and γ-glutamylcysteine synthetases by α-alkyl analogs of methionine sulfoximine that induce convulsions. J. Biol. Chem. 253:2333-2338.
9. Proler, M. and P. Kellaway. 1962. The methionine sulfoximine syndrome in the cat. Epilepsia. 3:117-130.
10. Gershoff, S. N., and C. A. Elvehjem. 1951. The relative effect of methionine sulfoximine on different animal species. J. Nutr. 45:451-458.
11. Newall, G. W., T. C. Erickson, W. E. Gilson, S. N. Gershoff, and C. A. Elvehjem. 1949. Studies on human subjects receiving highly agenized food materials. J. Clin. Lab. Invest. 34:239-245.
12. Griffith, O. W., 1982. Mechanism of action, metabolism, and toxicity of buthionine sulfoximine and its higher homologs, potent inhibitors of glutathione biosynthesis. J. Biol. Chem. 257:13704-1 3712.
13. Cooper, A. J. L., R A. Stephani, and A. Meister. 1976. Enzymatic reactions of methionine sulfoximine. Conversion to the corresponding α-imino and α-keto acids and to α-ketobutyrate and methane sulfinimide. J. Biol. Chem. 251:6674-6682.
14. Horwitz, M. A., Harth, G., Dillon, B. J. and Maslega-Galic, S. 2000. Recombinant BCG vaccines expressing the Mycobacterium tuberculosis 30 kDa major secretory protein induce greater protective immunity against tuberculosis than conventional BCG vaccines in a highly susceptible animal model. Proc. Natl. Acad. Sci. USA. 97:13853-13858.
15. Griffith, O. W., Han, J. and Martensson, J. 1991. Vitamin C protects adult guinea pigs against tissue damage and lethality caused by buthionine sulfoximine-mediate glutathione depletion. FASEB J. 5:A1182.
16. Griffith, O. W., Anderson, M. E., and Meister, A. 1979. Inhibition of glutathione biosynthesis by prothionine sulfoximine (S-n-Propyl-Homocysteine Sulfoximine), a selective inhibitor of γ-glutamylcysteine synthetase. J. Biol. Chem. 254:1205-1210.
17. Griffith, O. W., and Meister, A. 1979. Potent and Specific Inhibition of Glutathione Synthesis by Buthionine Sulfoximine (S-n-Butyl-Homocysteine Sulfoximine). J. Biol. Chem. 254: 7558-7560.
18. Tokutake, N., Hiratake, J., Katoh, M., Irie, T., Kato, H. and Oda, J. 1998. Design, Synthesis and Evaluation of Transition-state Analogue Inhibitors of Escherichia coli γ-Glutamylcysteine Synthetase. Biorganic Med. Chem. 6,1935-1953.
19. Liaw, S. H., and Eisenberg, D. 1994. Structural Model for the Reaction Mechanism of Glutamine Synthetase, Based on Five Crystal Structures of Enzyme-Substrate Complexes. Biochemistry 33:675-681.
20. Gill, H. S. and Eisenberg, D. 2001. The crystal structure of phosphinothricin in the active site of glutamine synthetase illuminates the mechanism of enzyme inhibition. Biochemistry 40:1903-1912.
21. Wolfenden, R. 1999. Conformational Aspects of Inhibitor Design: Enzyme-Substrate Interactions in the Transition State. Bioorganic Med. Chem. 7:647-552.
22. Schramm, V. L. 1998. Enzymatic Transition States and Transition State Analog Design. Ann. Rev. Biochem. 67:693-720.
23. Manning, J M., Moore, S., Rowe, W. B. and Meister, A. 1969. Identification of L-Methionine-S-sulfoximine as the Diastereomer of L-Methionine-SR-sulfoximine that Inhibits Glutamine Synthetase. Biochemistry 8:2681-2685.
24. Fadel, A. and Salaun, J. 1987. α-Alkylation of Acyclic Amino Acids with Self-reproduction of the Center of Chirality. A new route to (S)-(+)-α-Alkylated Aspartic Acids. Tetrahedron Lett. 28,2243-2246.
25. Rowe, W. B. and Meister, A. 1970. Identification of L-Methionine-S-Sulfoximine as the Convulsant Isomer of Methionine Sulfoximine. Proc. Natl. Acad. Sci. USA. 66: 500-506.
26. Campbell, E. B., Hayward, M. L., and Griffith, O. W. 1991. Analytical and Preparative Separation of Diastereomers of L-Buthionine-SR-Sulfoximine, a Potent Inhibitor of Glutathione Biosynthesis. Anal. Biochem. 194: 268-277.
27. Tate, S. S. and Meister, A. 1973. Glutamine Synthetases of Mammalian Liver and Brain. In The Enzymes of Glutamine Metabolism (S. Prusiner and E. R. Stadtman, eds.) Academic Press, New York. pp. 77-127.
28. Chamberlin, A. R., Koch, H. P., and Bridges, R. J. 1998. Design and synthesis of conformationally constrained inhibitors of high-affinity, sodium dependent glutamate transporters. Meth. Enzymol. 296:175-189.
29. Logusch, E. W., Walker, D. M., McDonald, J. F., and Franz, J. E. 1990. Inhibition of Escherichia coli glutamine synthetase by α- and γ-substituted phosphinothricins. Biochemistry 29:366-372.
30. Logusch, E. W., Walker, D. M., McDonald, J. F., and Franz, J. E. 1989. Substrate variability as a factor in enzyme inhibitor design: Inhibition of ovine brain glutamine synthetase by α- and γ-substituted phosphinothricins. Biochemistry 28:3043-3051.
31. Nak-aki, T., Mishima, A., Suzuki,. E., Shintani, F., and Fujii, T. 2000. Glufosinate ammonium stimulates nitric oxide production through N-methyl D-aspartate receptors in rat cerebellum. Neurosci. Lett. 290:209-212.
32. Matsumura, N., Takeuchi, C., Hishikawa, K., Fujii, F., and Nakaki, T. 2001. Glufosinate ammonium induces convulsions through N-methyl-D-aspartate receptors in mice. Neurosci. Lett. 304:123-125
33. Takahashi, H., Toya, T., Matsumiya, N., and Koyama, K. 2000. A case of transient diabetes insipidus associated with poisoning by a herbicide containing glufosinate. Clin Toxicol. 38:153-156.
34. Hoerlein, G. 1994. Glufosinate (phosphothricin), a natural amino acid with unexpected herbicidal properties. Rev. Environ. Contamin. Toxicol. 138:73-1 45.
35. Bartsch, K., Dichmnann, R., Schmitt, P., Uhlmann, E., and Schulz, A. 1990. Stereospecific production of the herbicide phosphinothricin (Glufosinate) by transamination: cloning, characterization, and overexpression of the gene encoding a phosphinothricin-specific transaminase in Escherichia coli. Appl. Environ Microbiol 56:7-12.
36. Eisenberg, D., Gill, H. S., Pfluegl, G. M. U., and Rotstein, S. H. 2000. Structure-function relationships of glutamine synthetases. Biochim. Biophys. Acta. 1477:122-1 45.
37. Listrom, C. D., Morizono, H., Rajagopal, B. S., McCann, M. T., Tuchmnan, M. and Allewell, N. M. 1997. Expression, Purification, and Characterization of Recombinant Human Glutamine Synthetase. Biochem. J. 328:159-163.
38. Tumani, H., Shen, G. Q., and Peter, J. B. 1995. Purification and immunocharacterization of human brain glutamine synthetase and its detection in cerebrospinal fluid and serum by sandwich enzyme immunoassay. J. Immunol. Meth. 188:155-163.
39. Misra, I. and Griffith, O. W. 1998. Expression and Purification of Human γ-Glutamylcysteine Synthetase, Prot. Exp. Purific. 13:268-276.
40. Abbott, J. J., Pei, J., Ford, J. L., Qi, Y., Grishin, V. N., Pitcher, L. A., Phillips, M. A., and Grishin, N. V. 2001. Structure Prediction and Active Site Analysis of the Metal Binding Determinants in γ-Glutamylcysteine Synthetase J. Biol. Chem. 276:42099-42107.
41. Gill H S, Pfluegl G M, Eisenberg D. 2002. Multicopy crystallographic refinement of a relaxed glutamine synthetase from Mycobacterium tuberculosis highlights flexible loops in the enzymatic mechanism and its regulation. Biochemistry. 41:9863-72.
42. Griffith, O. W. 1987. Amino Acid Sulfoximines: α-Ethylmmethionine Sulfoximine. Meth. Enzymol. 143:286-291.
43. Fadel, A. and Salaun, J. 1987. α-Alkylation of Acyclic Amino Acids with Self-reproduction of the Center of Chirality. A new route to (S)-(+)-α-Alkylated Aspartic Acids. Tetrahedron Lett. 28, 2243-2246.
44. Griffith, O. W. and Campbell, E. B. 1987. Resolution of Cysteine and Methionine Enantiomers. Meth. Enzymol. 143:166-172.
45. Campbell, E. B., Hayward, M. L., and Griffith, O. W. 1991. Analytical and Preparative Separation of Diastereomers of L-Buthionine-SR-Sulfoximine, a Potent Inhibitor of Glutathione Biosynthesis, Anal. Biochem. 194:268-277.

What is claimed is:

1. An anti-mycobacterial composition comprising a mycobacterial glutamine synthetase (MbGS) inhibitor of Formula 1: 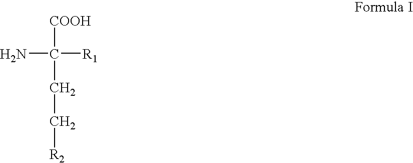
wherein:
R₁=branched and straight-chain alkyl groups of 1 to 8 carbons, and
R₂=tetrahedral group selected from the group consiting of: 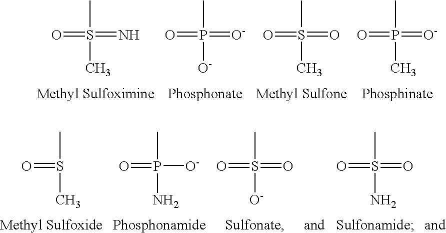
wherein said anti-mycobacterial composition effectively inhibits MbGS but does not substantially inhibit mammalian glutamine synthetase (MGS) in vivo.

2. The anti-mycobacterial composition according to claim 1 wherein said R₁ is branched and straight-chained alkyl groups of from two to four carbons.

3. An anti-mycobacterial composition comprising alpha-methyl-D,L-methionine-SR-sulfoxamine (α-Me-MSO) or alpha-ethyl-D,L-methionine-SR-sulfoxamine (α-Et-MSO) wherein said anti-mycobacterial composition effectively inhibits MbGS but does not substantially inhibit mammalian glutamine synthetase (MGS) in vivo.

4. An anti-mycobacterial composition comprising alpha-methyl-L-methionine-S-sulfoxamine (α-Me-MSO) or alpha-ethyl-L-methionine-S-sulfoxamine (α-Et-MSO) wherein said anti-mycobacterial composition effectively inhibits MbGS but does not substantially inhibit mammalian glutamine synthetase (MGS) in vivo.

5. A method for treating, palliating or inhibiting mycobacterial infections in a mammal comprising:
administering to a mammal having a mycobacterial infection an anti-microbial effective amount of an anti-mycobacterial composition comprising gamma-substituted alpha-amino-alpha-alkyl-butyrates that effectively inhibit mycobacterial glutamine synthetase (MbGS), but do not substantially interfere with mammalian glutamine synthetase (MGS) in vivo such that said mycobacterial infection is treated, palliated or inhibited.

6. The method for treating mycobacterial infections in a mammal according to claim 5 wherein said administering step further comprises said gamma-substituted alpha-amino-alpha-alkyl-butyrate wherein said alpha alkyl group is branched and straight-chained alkyl groups from 2 to 8 carbons and said gamma substituent is a tetrahedral sulfur or phosphorus group.

7. The method for treating mycobacterial infections in a mammal according to claim 6 wherein said alpha alkyl group is branched and straight-chained alkyl groups from 2 to 4 carbons.

8. The method for treating mycobacterial infections in a mammal according to claim 6 wherein said tetrahedral sulfur group is selected from the group consisting of methyl sulfoximine, methyl sulfone, methyl sulfoxide, sulfonate, and sulfonamide

9. The method for treating mycobacterial infections in a mammal according to claim 6 wherein said tetrahedral phosphorus group is selected from the group consisting of phosphonate, methylphosphinite, phosphonamide.

10. A method for treating, palliating or inhibiting mycobacterial infections in a mammal comprising:
administering to a mammal having a mycobacterial infection an anti-microbial effective amount of an anti-mycobacterial composition comprising alpha-methyl-L-methionine-S-sulfoxamine (α-Me-MSO) or alpha-ethyl-L-methionine-S-sulfoxamine (α-Et-MSO) wherein said anti-mycobacterial composition effectively inhibits MbGS but does not substantially inhibit mammalian glutamine synthetase (MGS) in vivo.

11. The method according to claim 5 further comprising co-administering an anti-microbial effective amount of isoniazid (INH).

12. The method for treating, palliating or inhibiting mycobacterial infections in a mammal according to any one of claims 5 to 11 wherein said mammal is selected from the group consisting of humans, monkeys, cows, pigs, horses, rabbits, rodents, cats and dogs.

13. The method for treating, palliating or inhibiting mycobacterial infections in a mammal according to any one of claims 5 to 11 wherein said mycobacterial infection is caused by a member of the genus Mycobacterium selected from the group consisting of M. tuberculosis, M. bovis, M. avium.

14. A method for treating, palliating or inhibiting mycobacterial infections in a mammal comprising:
co-administrating and anti-mycobacterial effective amount of L-methionine-SR-sulfoximine (MSO) and ascorbic acid.
